(19) United States
(12) Patent Application Publication
(10) Pub. No.: US 2006/0074450 A1
(43) Pub. Date: Apr. 6, 2006

(54) System for providing electrical pulses to nerve and/or muscle using an implanted stimulator

(76) Inventors: Birinder R. Boveja, Milwaukee, WI (US); Angely Widhany, Milwaukee, WI (US)

Correspondence Address: BIRINDER R. BOVEJA & ANGELY WIDHANY, P. O. BOX 210095, MILWAUKEE, WI, 53221, US

(21) Appl. No.: 11/234,337

(22) Filed: Sep. 23, 2005

Related U.S. Application Data
(63) Continuation-in-part of application No. 10/436,017, filed on May 11, 2003.

(51) Int. Cl. A61N 1/18

(52) U.S. Cl. 607/2

(57) ABSTRACT

Implantable stimulation systems and method for nerve and/or muscle stimulation applications comprises two functional modules which are, i) a programmable pulse generator module, and ii) a stimulus-receiver module. The stimulus-receiver module is designed to provide stimulation/blocking pulses with an external stimulator. An external device acts as a programmer and as an external stimulator. The system uses implantable power source until stable external power is available. A power select circuitry switches between implanted power source and external power source, when its available. Stimulation/blocking to nerve or muscle tissue may be provided using implantable pulse generator, or via an external stimulator which is inductively coupled to the stimulus-receiver portion of the implanted system. Numerous applications of the system include, spinal cord stimulation to provide therapy for intractable pain and refractory angina; occipital nerve stimulation to provide therapy for occipital neuralgia and transformed migraine; afferent vagus nerve modulation to provide therapy for a host of neurological and neuropsychiatric disorders such as epilepsy, depression, Parkinson's disease, bulemia, anxiety/obsessive compulsive disorders, Alzheimer's disease, autism, and neurogenic pain; efferent vagus nerve stimulation for rate control in atrial fibrillation, and to provide therapy for congestive heart failure; gastric nerves or gastric wall stimulation to provide therapy for obesity; sacral nerve stimulation to provide therapy for urinary urge incontinence; deep brain stimulation to provide therapy for Parkinson's disease, and other neurological and neuropsychiatric disorders; cavernous nerve stimulation to provide therapy for erectile dysfunction.

This application is a continuation of application Ser. No. 10/436,017 filed May 11, 2003, entitled “Method and system for providing pulsed electrical stimulation to a cranial nerve of a patent to provide therapy for neurological and neuropsychiatric disorders”.

### FIELD OF INVENTION

This invention relates to implantable stimulation systems, more particularly to nerve or muscle stimulation system capable of being used as a programmable implantable pulse generator, or as an implanted stimulus-receiver used in conjunction with an external stimulator.

### BACKGROUND

Implantable pulse generators are known in the art for various nerve and muscle stimulation applications. Some examples of nerve and muscle stimulation applications are, without limitation, spinal cord stimulation to provide therapy for intractable pain and refractory angina; occipital nerve stimulation to provide therapy for occipital neuralgia and transformed migraine; afferent vagus nerve modulation to provide therapy for a host of neurological and neuropsychiatric disorders such as epilepsy, depression, Parkinson's disease, bulimia, anxiety/obsessive compulsive disorders, Alzheimer's disease, autism, and neurogenic pain; efferent vagus nerve(s) stimulation for rate control in atrial fibrillation, and to provide therapy for congestive heart failure; gastric nerves or gastric wall stimulation to provide therapy for obesity; sacral nerve stimulation to provide therapy for urinary urge incontinence; deep brain stimulation to provide therapy for Parkinson's disease, and other neurological disorders; cavernous nerve stimulation to provide therapy for erectile dysfunction; phrenic nerve stimulation or diaphragmatic pacing to help with breathing; and functional electrical stimulation of muscles.

Unlike cardiac pacing, where the implanted pulse generator (IPG) is mostly in the monitoring mode, nerve and muscle stimulation applications mentioned above can be quite demanding on the battery (power source), due to the frequency of stimulation pulses provided to the nerve or muscle tissue, to provide optimal therapy. Because of this, many applications in the past have utilized an external stimulator in conjunction with an implanted stimulus-receiver. In such systems, a primary coil from an external stimulator is inductively coupled to an implanted secondary coil of the stimulus-receiver. The implanted stimulus-receiver may be a passive device, or may comprise a temporary power source, such as a high-value capacitor. Nerve stimulation/modulation utilizing a passive stimulus-receiver is disclosed in Applicant's U.S. Pat. No. 6,205,359. An implanted stimulus-receiver with temporary power source is disclosed in Applicant's patent application Ser. No. 10/196,533. Both of the above disclosures are incorporated herein by reference.

Both, implanted pulse generator (IPG) and inductively coupled systems have unique advantages that makes them suitable for certain applications. For many conventional indications, as well as, emerging indication, an ideal system would be a device which can be used as an implanted stimulus-receiver, or as a fully programmable implanted pulse generator. Such a system is disclosed in this application. Some of the advantages of systems disclosed here include:

Regulated pulses will be delivered to nerve or muscle tissues using either battery or external power;

Unlimited telemetry without battery drain; and

Lower voltage levels required at the receiving coil.

A block diagram of a representative prior art implantable pulse generator (IPG) 391 is shown in conjunction with FIG. 1. A microcontroller 398 controls the output pulses delivered to the nerve or muscle tissues 54, based on the programs stored in the memory. Predetermined program (comprising pulse parameters) is stored in the memory via an external programmer 85. Further, individual parameters may also be adjusted non-invasively via the external programmer 85. Communication with the external programmer 85, occurs via a coil (inductor) 383 in the programmer 85 and a coil 399 within the IPG 391. Typically, a hermetically sealed lithium battery 397 is used for providing power to all components within the IPG 391. The service life of a non-rechargeable battery unit may be only 1-3 years, after which the IPG 391 would have to be surgically explanted and replaced with another unit.

Implanted stimulus-receiver used in conjunction with an external stimulator are used because the battery life is not crucial in an external stimulator. The implanted stimulus-receiver will theoretically last for a long time. These are known in the art as “RF system”. The RF systems are ideally suited for applications where high intensity is generally required for short periods of time. Numerous nerve and muscle stimulation applications fall into this category. One example is spinal cord stimulation for patients who have high thresholds. Another example is stimulation of fascia around the occipital nerves, to provide therapy for occipital neuralgia and transformed migraines.

Implanted pulse generator (IPG) systems are ideally suited for applications where some low level intermittent baseline stimulation is required on a continual basis and the therapy is cumulative, such as with afferent vagal nerve stimulation for neurological and neuropsychiatric disorders.

For many nerve and muscle stimulation applications, a system that comprises both a stimulus-receiver module and a programmable IPG module would be an ideal system.

### PRIOR ART

Prior art search reveals either inductively coupled systems for nerve or muscle stimulation, or rechargeable implantable pulse generator (IPG). Applicant's invention is different from a rechargeable IPG, in that in the current invention, the electrical stimulation may be provided via the IPG or may be provided via an external stimulator.

U.S. Pat. No. 6,205,359 (Boveja), U.S. Pat. No. 6,208,902 (Boveja), and U.S. Pat. No. 6,505,074 (Boveja) are generally directed to an inductively coupled system for providing stimulation to nerve tissue to provide therapy.

U.S. Pat. No. 6,576,227 (Meadows et al.) and U.S. Pat. No. 6,895,280 (Meadows et al.) are generally directed to rechargeable spinal cord stimulator systems. U.S. Pat. No. 6,941,171 (Mann et al.) is generally directed to systems for incontinence and pain.

### SUMMARY OF THE INVENTION

Prior art nerve or muscle stimulators are generally either an external stimulator with implanted stimulus-receiver, or a stand-alone implanted pulse generator (IPG) comprising an implanted battery power source. Both types of systems have unique advantages.

Novel systems and method of the current invention comprises advantages of both types of systems, i.e. “RF” systems and IPGs.

Accordingly, in one aspect of the invention an implantable stimulator for providing electrical pulses to nerve or muscle tissue comprises, a programmable implantable pulse generator means and a stimulus-receiver means, wherein the stimulus-receiver means is adapted to function in conjunction with an external stimulator.

In another aspect of the invention, electrical pulses to nerve and muscle tissue are provided utilizing an implanted power source.

In another aspect of the invention, electrical pulses are provided from an external power source.

In another aspect of the invention, electrical pulses are provided from an external stimulator which is inductively coupled to an implanted stimulus-receiver means.

In another aspect of the invention, the implanted stimulator comprises circuitry means for switching between implanted power source (battery), or external power source when stable external power is available.

In another aspect of the invention, implanted stimulator comprises non-rechargeable battery.

In another aspect of the invention, implanted stimulator comprises a rechargeable battery.

In another aspect of the invention, the implanted stimulator comprises a coil which is inside a titanium case.

In another aspect of the invention, the implanted stimulator comprises a coil which is outside a titanium case.

In another aspect of the invention, the implanted stimulator comprises a coil which is outside a titanium case, and on the titanium case with a magnetic shield between the coil and the titanium case.

In another aspect of the invention, the implanted stimulator comprises a coil which is external and around a titanium case.

In another aspect of the invention, the system can be used for providing electrical pulses to spinal cord of a patient to provide therapy or alleviate symptoms for at least one of intractable pain or refractory angina.

In another aspect of the invention, the system can be used for providing electrical pulses to occipital nerves or branches of a patient, to provide therapy or alleviate symptoms for at least one of occipital neuralgia or transformed neuraliga.

In another aspect of the invention, the system can used for providing electrical pulses to a vagus nerve(s) of a patient for afferent vagus stimulation, to provide therapy or alleviate symptoms for at least one of epilepsy, depression, Parkinson's disease, bulimia, anxiety/obsessive compulsive disorders, Alzheimer's disease, autism, and neurogenic pain.

In another aspect of the invention, the system can be used for providing electrical pulses to a vagus nerve(s) of a patient for efferent vagus stimulation, for rate control in atrial fibrillation, or to provide therapy for congestive heart failure.

In another aspect of the invention, the system can be used for providing electrical pulses to gastric nerves or gastric wall of a patient to provide therapy for obesity.

In another aspect of the invention, the system can be used for providing electrical pulses to sacral nerves or branches of a patient, to provide therapy or alleviate symptoms for at least one of intractable pain or refractory angina.

In another aspect of the invention, the system can used for deep brain stimulation of a patient to provide therapy or all alleviate symptoms for neurological or neurospsychiatric disorders including Parkinson's disease.

In yet another aspect of the invention, the system can be used for providing electrical pulses to cavernous nerve(s) or branches of a patient, to provide therapy for erectile dysfunction.

Various other features, objects and advantages of the invention will be made apparent from the following description taken together with the drawings.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a block diagram of a representative prior art programmable implantable pulse generator (IPG).

FIG. 2 depicts an implantable stimulator with two functional modules.

FIG. 3 is a block diagram of the implantable stimulator with non-rechargeable battery.

FIG. 4 is a block diagram of the implantable stimulator with a rechargeable battery.

FIG. 5 is a block diagram depicting battery recharging circuitry.

FIG. 6 is a diagram depicting the power source select circuit of the implantable stimulator.

FIG. 7 is a circuit of a pulse width modulation (PWM) voltage regulator.

FIG. 8A is a block diagram of the isolation circuit.

FIGS. 8B and 8C are diagrams showing two forms of isolation circuitry.

FIG. 9 is a diagram depicting externalization of the coil via two separate feed-throughs.

FIG. 10 is a diagram depicting externalization of the coil via one separate feed-through.

FIG. 11 is a diagram depicting externalization of the coil via two feed-throughs which are common with the feed-throughs for the lead.

FIG. 12 is a diagram depicting externalization of the coil via one feed-through which is common with the feed-through for the lead.

FIG. 13 is a simplified diagram depicting externalizing the coil.

FIG. 14A is a diagram showing an externalized coil on titanium case.

FIG. 14B is an exploded view of an externalized coil, showing the placement of a magnetic shield in-between the coil and the titanium case.

FIG. 15 is a schematic diagram showing two paths for inductively received energy.

FIG. 16A depicts a bipolar configuration of the stimulus-receiver portion.

FIG. 16B depicts a unipolar configuration of the stimulus-receiver portion.

FIG. 17 depicts a simplified flow diagram of the stimulator system.

FIG. 18 depicts a flow diagram of the implantable stimulator system, for a non-rechargeable battery version.

FIG. 19 is a diagram of a pulse generator output circuit using a charge pump.

FIG. 20 is a flow diagram of the system software flow when a pulse is called for.

FIG. 21A is a diagram depicting a coil externalized from the titanium case, and positioned in the header.

FIG. 21B is diagram of a representative lead.

FIG. 22 depicts an application of the system for spinal cord stimulation.

FIG. 23 depicts a cross section of the spinal cord showing pain transmission neurons.

FIGS. 24A and 24B depict placement of a pair of leads with electrodes adjacent to occipital nerves.

FIG. 25 depicts a stimulation system for providing stimulation to occipital nerves.

FIG. 26 depicts an application of the stimulation system for providing electrical pulses to left vagus nerve for central nervous system (CNS) applications.

FIG. 27 depicts an application of the system for providing deep brain stimulation.

FIGS. 28 and 29 depicts an application of the system for providing vagal stimulation/blocking to provide therapy for obesity.

FIG. 30 depicts an application of the system for sacral nerve(s) stimulation to provide therapy for urinary incontinence.

FIG. 31 depicts an application of the system to provide therapy for erectile dysfunction.

FIGS. 32 and 33 depict an application of the system to provide electrical pulses to gastric wall of a patient to provide therapy for obesity.

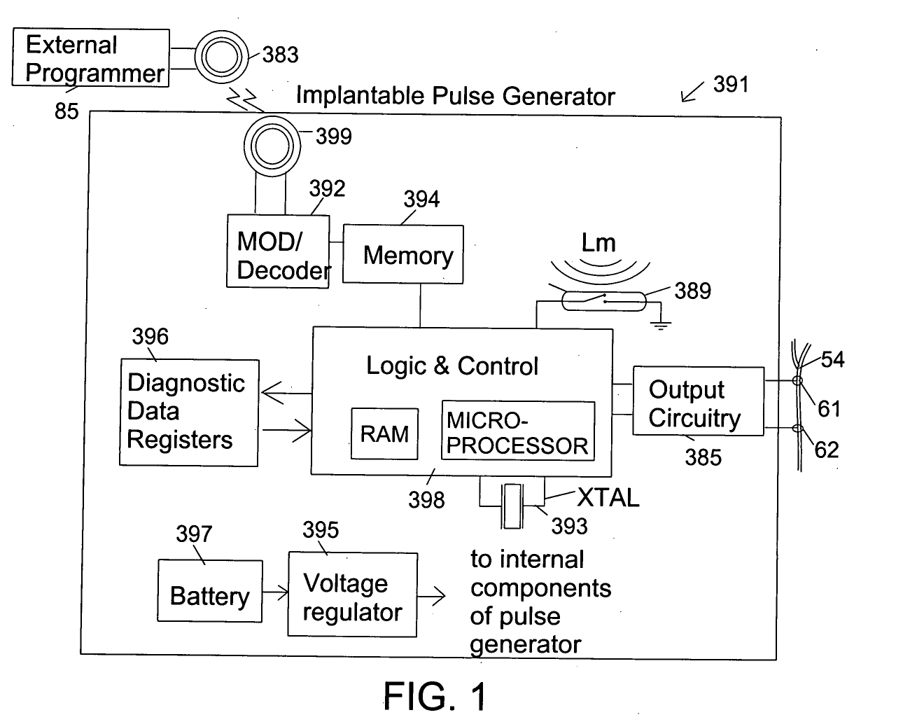

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

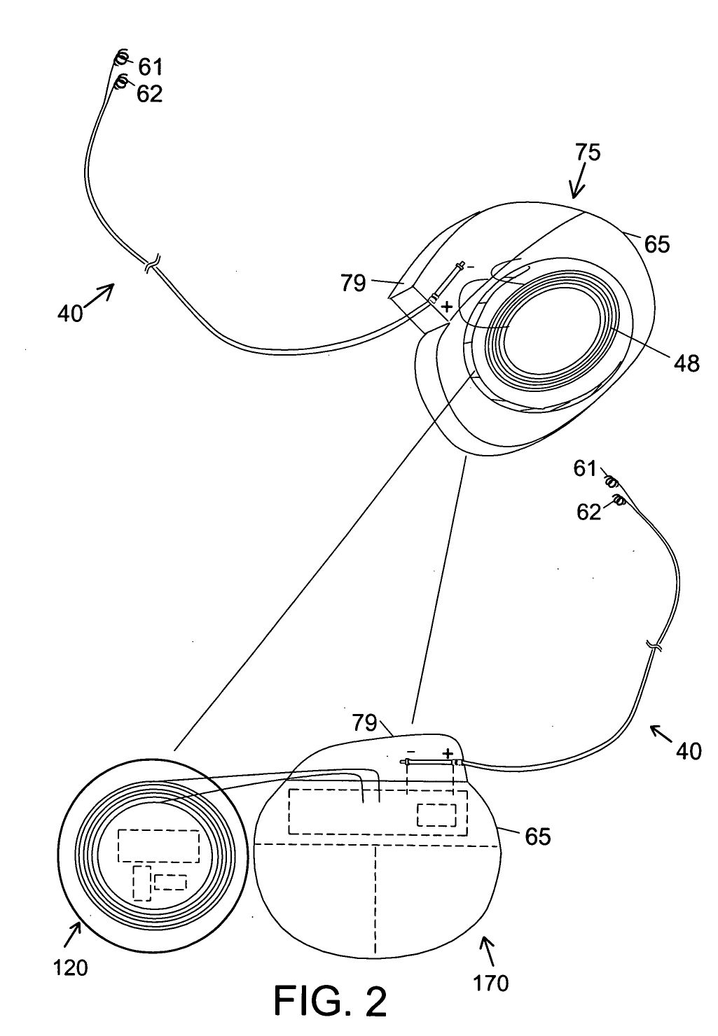

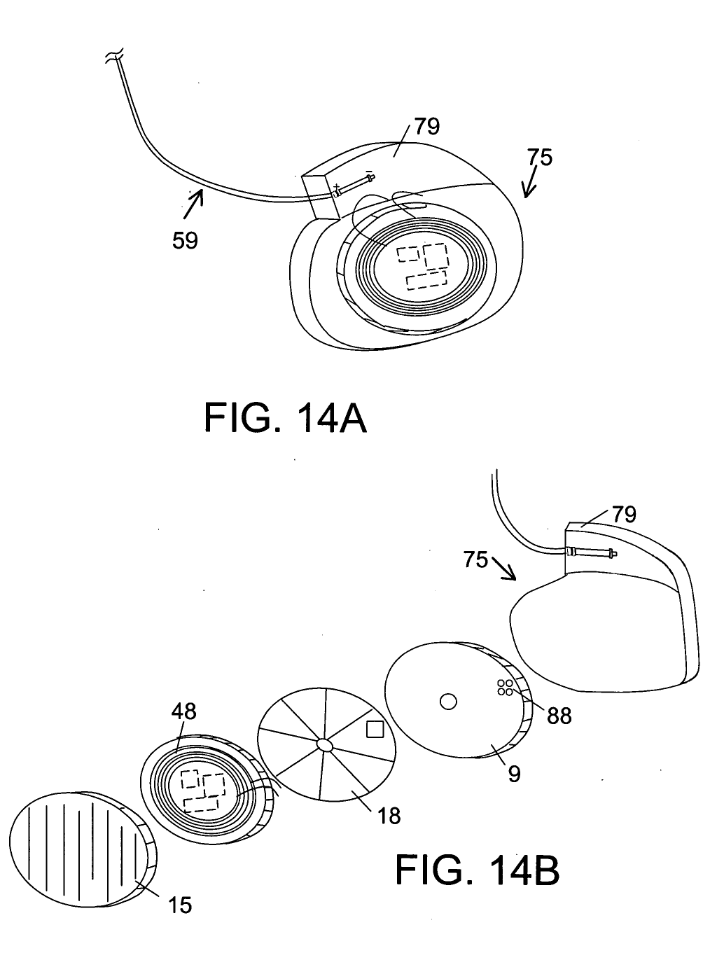

Depicted in FIG. 2 is one embodiment of an implantable stimulator 75, which comprises two functional modules, i) a programmable implantable pulse generator, and ii) a stimulus-receiver module which is used with an external stimulator/pulse generator. In this embodiment, the electronic circuitry is encased in a hermetically sealed titanium can, as is well known in the art. A secondary coil 48, which is used for inductively receiving pulses, or for programming may be outside a titanium case, around the titanium case, or may be inside the titanium case. If the coil, is outside and on the titanium case, a magnetic shield is placed between the titanium case and the externalized (secondary) coil 48, as is shown later in conjunction with FIG. 14B.

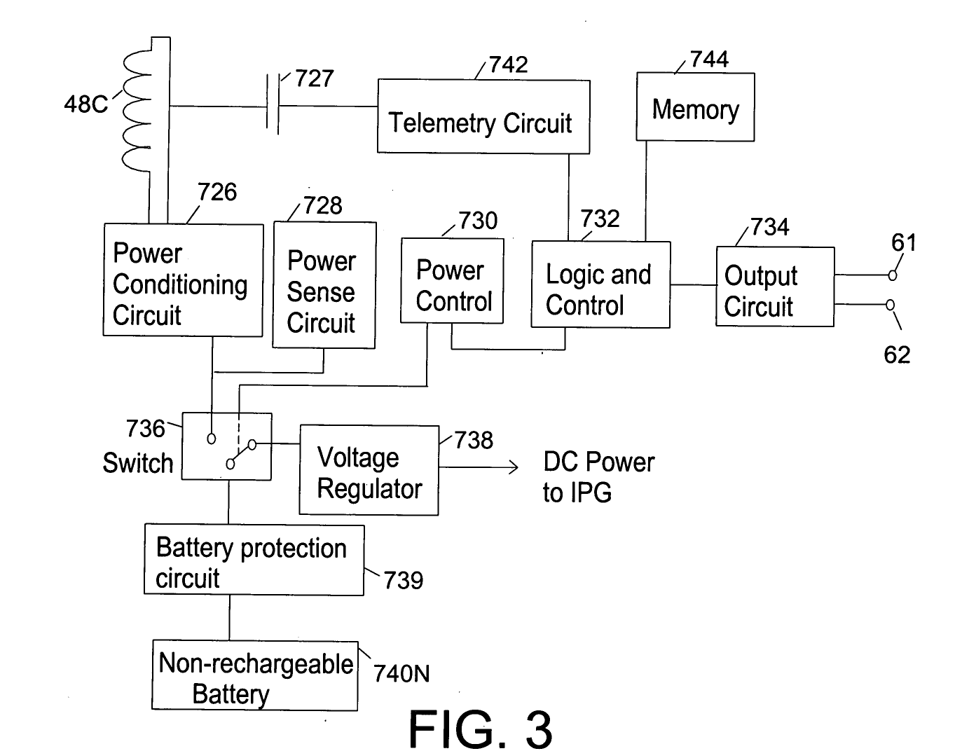

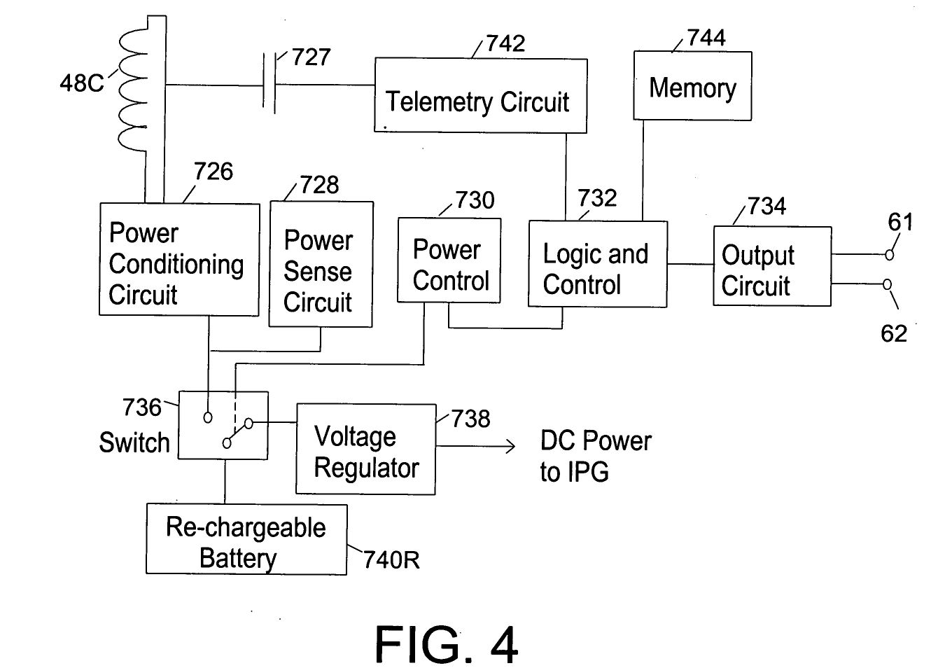

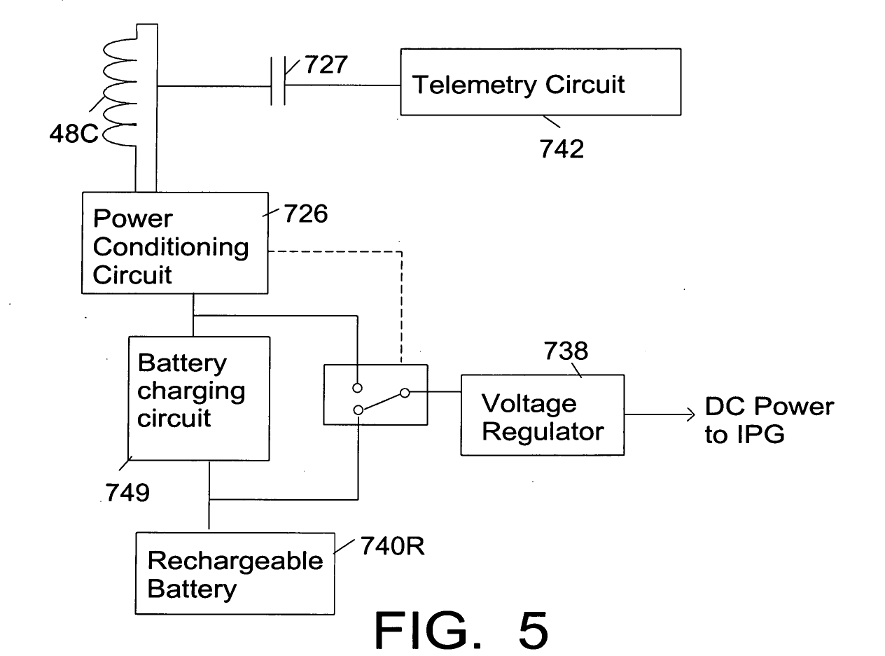

Shown in conjunction with FIGS. 3 and 4 is a simplified overall block diagram of one embodiment of the invention. A coil 48C which is external to the titanium case is used both as a secondary coil of a stimulus-receiver, and is also used as the forward and back telemetry coil for the implanted pulse generator (IPG). This embodiment may be practiced with a non-rechargeable battery 740N (FIG. 3), as well as, with a rechargeable battery 740R (FIG. 4). As shown in conjunction with FIG. 3, if a non-rechargeable battery 740N is used, a battery protection circuit 739 is provided within the system. Shown in conjunction with FIG. 4 is an embodiment comprising a rechargeable battery 740R, which is the preferred embodiment. The circuitry in the two versions are similar except for the battery charging circuitry 749, which is shown in conjunction with FIG. 5. This circuit is energized when external power is available. It senses the charge state of the battery 740R and provides appropriate charge current to safely recharge the battery without overcharging.

As also shown in conjunction with FIG. 4, the system comprises a power sense circuit 728 that senses the presence of external power communicated with the power control 730, when adequate and stable power is available from an external source. The power control circuit 730 controls a switch 736 that selects either battery power 740 or conditioned external power from 726 (not shown). The logic and control section 732 and memory 744 includes the IPG's microcontroller, pre-programmed instructions, and stored changeable parameters. Using input from the telemetry circuit 742 and power control 730, this section controls the output circuit 734 that generates the output pulses. The memory may also comprise predetermined/pre-packaged programs, which are programmed into the memory via an external programmer.

In this embodiment, the IPG circuitry within the titanium case is used for all stimulation pulses, whether the energy source is the internal battery 740 or an external power source. The external device serves as a source of energy, and as a programmer that sends telemetry to the IPG. For programming, the energy is sent as high frequency sine waves with superimposed telemetry wave driving the external coil 46C (not shown). The telemetry is passed through coupling capacitor 727 to the IPG's telemetry circuit 742. For pulse delivery using external power source, the stimulus-receiver portion will receive the energy coupled to the implanted coil 48C, and using the power conditioning circuit 726, rectify it to produce DC, filter and regulate the DC, and couple it to the IPG's voltage regulator 738 section so that the IPG can run from the externally supplied energy rather than the implanted battery 740.

External stimulators which are adapted to work in conjunction with implanted stimulus-receivers are known in the art. One such external stimulator is disclosed in Applicant's U.S. Pat. No. 6,366,814 (Boveja et al.) and is incorporated herein by reference.

The electrical parameters which can be individually programmed, include variables such as pulse amplitude, pulse width, frequency of stimulation, stimulation on-time, and stimulation off-time. Complex electrical pulses can also be provided. Complex electrical pulses comprises pulses which are configured to be one of non-rectangular, multi-level, biphasic, or pulses with varying amplitude during the pulse. The methodology for generating complex electrical pulses is well known in the art. Table one below defines the approximate range of parameters.

TABLE 1
Electrical parameter range that can be
provided to the nerve or muscle tissue

| PARAMER | RANGE |
|---|---|
| Pulse Amplitude | 0.1 Volt-25 Volts |
| Pulse width | 20 μS-5 mSec. |
| Stim. Frequency | 5 Hz-200 Hz |
| Freq. for blocking | DC to 750 Hz |
| On-time | 5 Secs-24 hours |
| Off-time | 5 Secs-24 hours |

For use of this system, in some applications a baseline level of stimulation is provided according to predetermined program stored in the memory 744 of the stimulator 75. Such baseline stimulation may be of a continuous-intermettent type. One example without limitation may be 5 min.-ON and 30 min.-OFF. Additionally, at selected times (such as during an aura), the stimulation may be supplemented with an external pulse generator, to a fast cycle rate. Advantageously, the energy intensive portion of the electrical stimulation is provided via an external power source. This would be useful for vagal afferent modulation to provide therapy or alleviate symptoms of neurological and neuropsychiatric disorders.

In other applications, for example for gastric myo-electric pacing therapy, the baseline continuous pacing may be turned off, and the patient may only use an external stimulator to provide pulses at around meal-times, or when the patient is hungry. At other times the baseline continuous-intermittent stimulation for the implanted power source may be turned on.

Different nerve and muscle stimulation applications are described later in this disclosure.

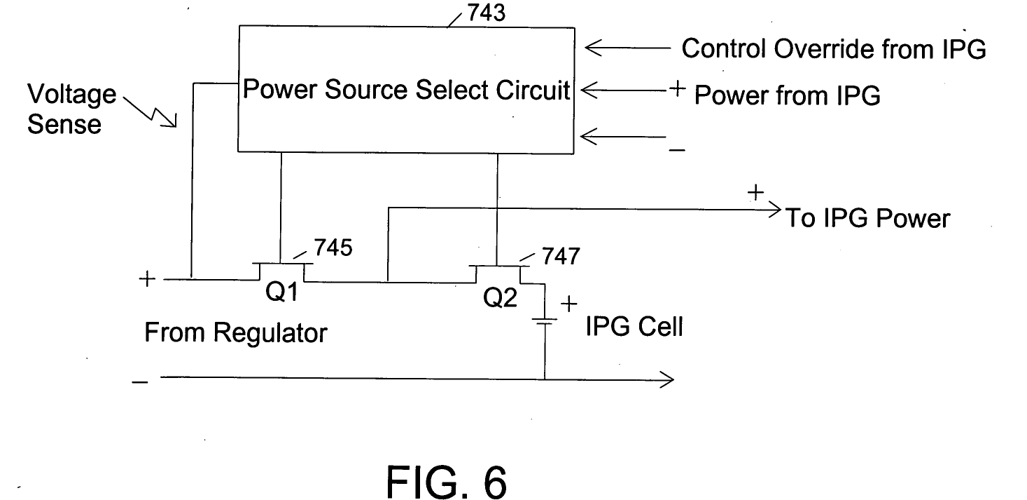

The power source select circuit is highlighted in conjunction with FIG. 6. The IPG provides stimulation pulses according to the stimulation programs stored in the memory 744 of the implanted stimulator 75, with power being supplied by the implanted battery 740. When stimulation energy from an external stimulator is inductively received via secondary coil 48C, the power source select circuit (shown in block 743) switch power via transistor Q1 745 and transistor Q2 747. Transistor Q1 and Q2 are preferably low loss MOS transistors used as switches, even though other types of transistors may be used.

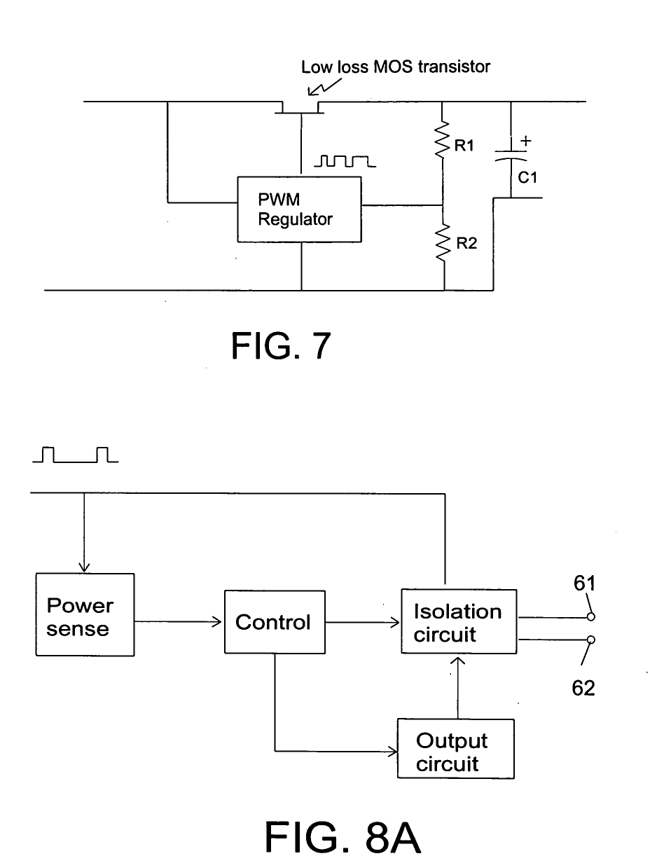

FIG. 7 shows a voltage regulator implemented using a pulse width modulation (PWM) regulator. The PWM regulator is a commercially available IC. The advantage when compared to a linear regulator using a pass transistor is that the PWM regulator is more efficient. In a linear regulator, the pass transistor limits the output voltage by increasing its resistance, thus creating a voltage drop across the transistor. Energy is wasted in the form of heat at a rate in Watts equal to the voltage drop across the transistor multiplied by the current in Amperes: For PWM regulator, the transistor acts as a switch that is either full-on or full-off. Thus either the current is near zero (off) or the voltage drop is small (on) and the heat loss is minimal. The output voltage is controlled by adjusting the duty cycle of the drive pulse train (on vs off time) so that just enough charge is delivered to the output capacitor and load to maintain the required voltage.

For the circuit in FIG. 7, this circuit regulates to output voltage from the half wave or full wave rectifier. The voltage divider formed by R₁and R₂ sets the desired output voltage across C₁. The regulator adjusts the duty cycle of the pulses applied to the gate of Q₁as needed to maintain the desired voltage across C₁. The circuit is more efficient than a pass-transistor regulator because Q₁ is either fully ON or fully OFF.

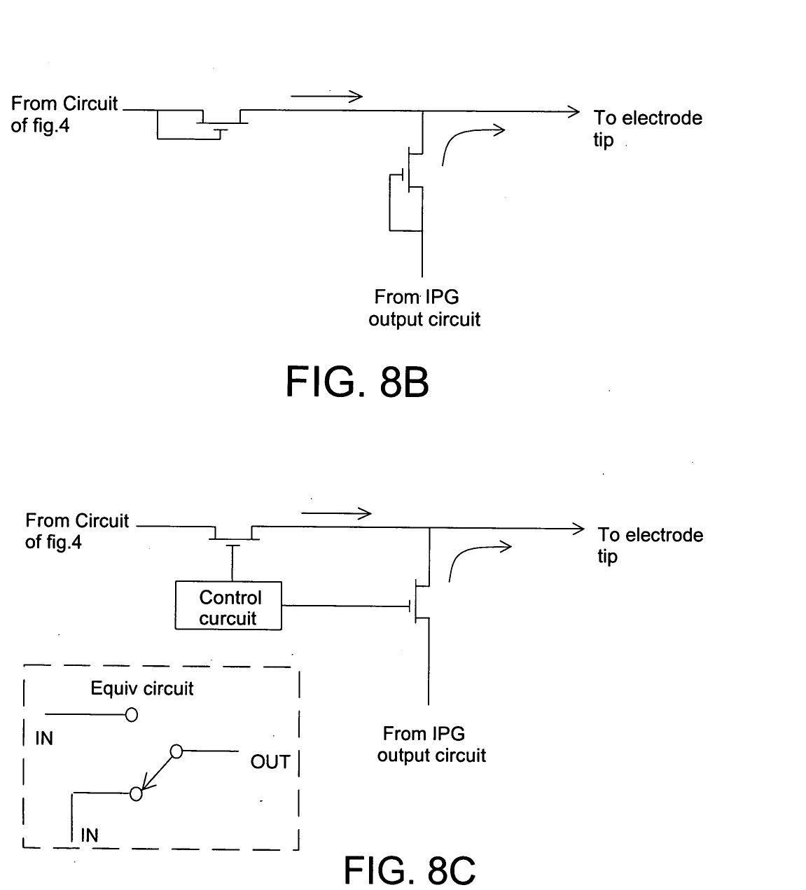

Because two different sources are available to provide pluses to the stimulation electrodes 61,62 the device provides a means to isolate the two sources. Otherwise, the pulse energy from one source would be divided between the stimulation electrode and the output device of the other source, thus wasting energy and reducing the stimulation voltage. FIG. 8A shows in block diagram form, a means of providing isolation between the output circuit of a conventional IPG and pulses coupled directly from an external source using a passive stimulus-receiver. As shown in this figure, the IPG's power sense detects the presence of an external pulse source and signals to control circuit. The control circuit energizes (turn on) the appropriate transistor to allow the pulses to pass to the lead electrodes, and keeps the other transistor off to disconnect the unneeded portion of the circuit.

FIGS. 8B and 8C, show passive and active means of isolating the two sources using field effect transistors acting as switches. In FIG. 8B, for the passive implementation, the transistors are wired in the “diode” connection: where they block current in one direction but allow current to pass in the other direction (indicated in the figure by arrows). For the passive circuit, the minimum stimulation pulse must be above the transistors turn-on threshold (typically 0.5 volts). In FIG. 8C, an active circuit controls the gate of each transistor and sets it to either the on or off state, as determined by the control circuitry. This guarantees full turn-on of the intended path even for very low stimulation levels.

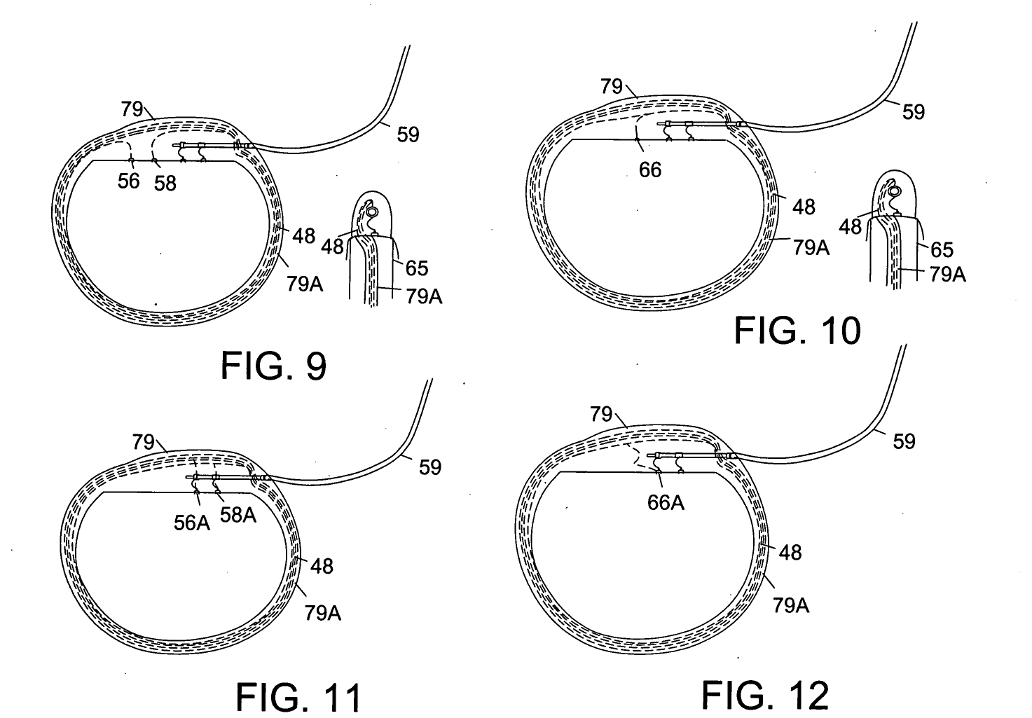

As was previously mentioned, the secondary coil 48 may be outside the titanium case 65, around the titanium case 65, or may be inside the titanium case 65. In one embodiment, as shown in conjunction with FIGS. 9-12, the coil 48 may be externalized at the header portion 79 of the implanted device, and may be wrapped around the titanium can. In this case, the coil is encased in the same material as the header 79. As shown in conjunction with FIGS. 9 and 10, the feed-through for the coil 56, 58 may be separate from the feed-through for the lead connection. Alternatively, as shown in conjunction with FIGS. 11 and 12, the feed-through of coil 48 may be combined with the feed-through for the lead.

FIGS. 10 and 12 show connection where one end of the coil 48 is connected to the exterior of the IPG's case. The circuit is completed by connecting the capacitor 729 and bridge rectifier 739 to the interior of the IPG's case. The advantage of this arrangement is that it requires one less hermetic feedthrough filter, thus reducing the cost and improving the reliability of the IPG. Hermetic feedthrough filters are expensive and a possible failure point. However, the case connection may complicit the output circuitry or limit its versatility. When using a bipolar electrode, care must be taken to prevent an unwanted return path for the pulse to the IPG's case. This is not a concern for unipolar pulses using a single conductor electrode because it relies on the IPG's case for return of the current pulse.

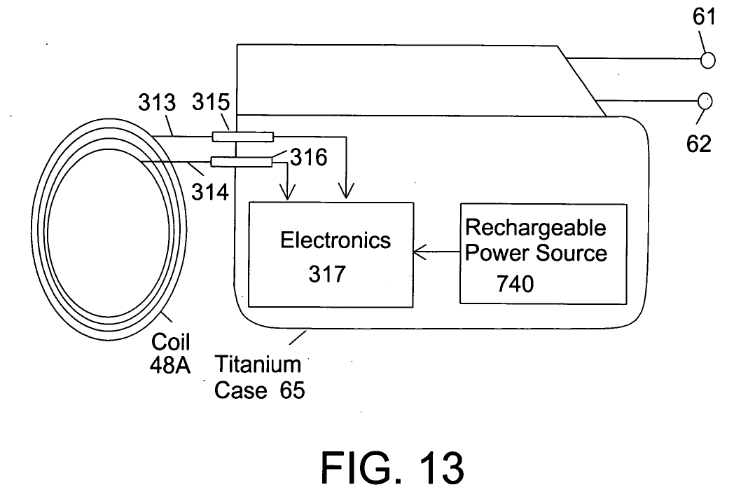

In an alternative embodiment, the coil may be positioned on the titanium case as disclosed in FIGS. 13, 14A and 14B. As shown in conjunction with FIGS. 14A and 14B, if the coil 48 is placed on the titanium case, a magnetic shield 18 is placed between the titanium case and the coil 48. The other components in FIG. 14B are a coil carrier 9 and a coil cover 15.

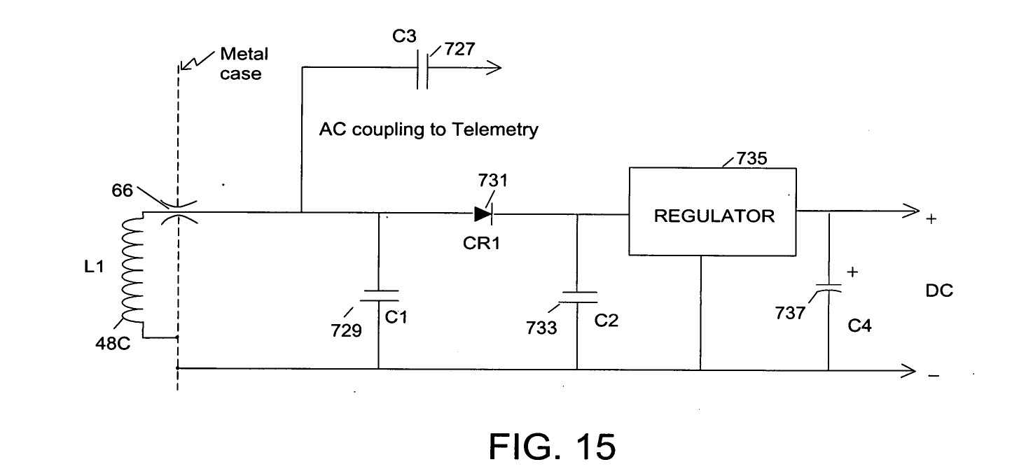

The stimulus-receiver portion of the circuitry is shown in conjunction with FIG. 15. Capacitor C1 (729) makes the combination of C1 (729) and L1 (48C) sensitive to the resonant frequency and less sensitive to other frequencies, and energy from an external (primary) coil 46C is inductively transferred to the implanted unit via the secondary coil 48C. The AC signal is rectified to DC via diode 731, and filtered via capacitor 733. A regulator 735 set the output voltage and limits it to a value just above the maximum IPG cell voltage. The output capacitor C4 (737), typically a tantalum capacitor with a value of 100 micro-Farads or greater, stores charge so that the circuit can supply the IPG with high values of current for a short time duration with minimal voltage change during a pulse while the current draw from the external source remains relatively constant. Also shown in conjunction with FIG. 15, a capacitor C3 (727) couples signals for forward and back telemetry.

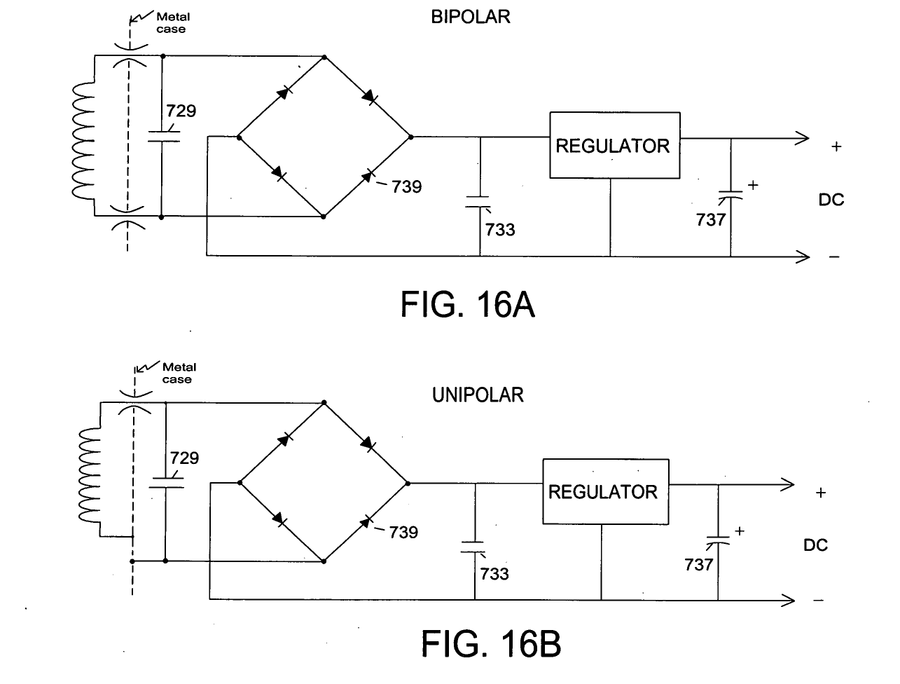

FIGS. 16A and 16B show schematics of stimulus-receiver in bipolar and unipolar configurations respectively. A diode bridge 739 has also been substituted in these figures for full wave rectification. In the unipolar configuration, a bigger tissue area is stimulated since the difference between the tip (cathode) and case (anode) is larger. Stimulation using both configurations is considered within the scope of this invention.

Even though for most nerve and muscle stimulation applications bipolar stimulation is the preferred mode, there are a few specific instances where unipolar stimulation is the preferred mode.

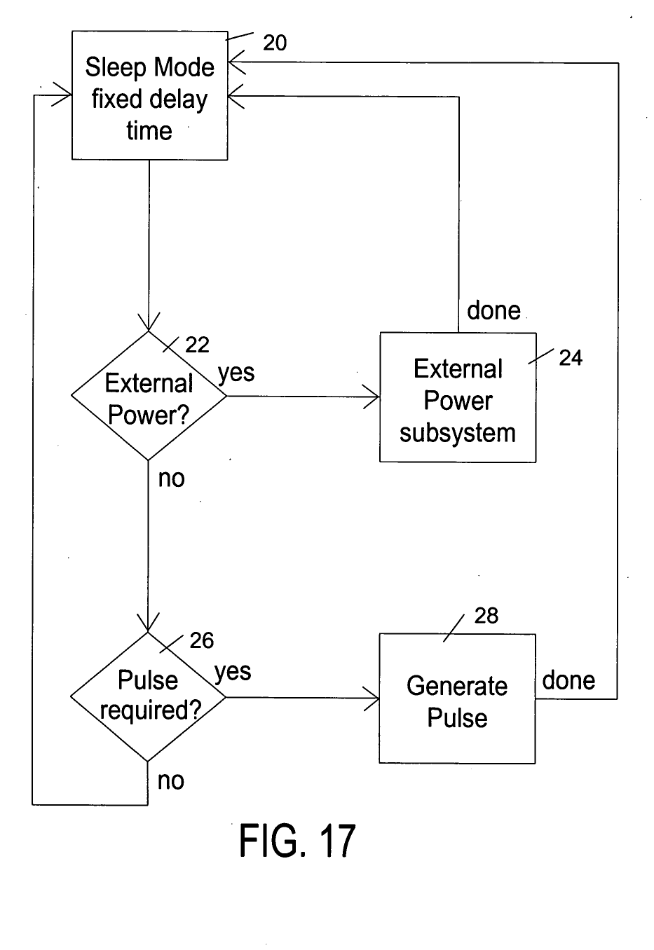

Shown in conjunction with FIG. 17 is a flowchart showing the main loop for the pulse generator's state machine. To conserve battery power, the pulse generator spends most of its time in sleep mode 20 where only the slow clock and sleep timeout counter is running. The slow clock increments the sleep timeout counter until a preset number of counts is reached, initiating the exit from sleep mode 20. The IPG starts the fast clock and begins running its main control program that first checks for the presence of the external power source 22 (indicated by an input line set active by the power supply hardware circuit). If external power 22 is present, the program passes control to the external power subsystem 24. The external power subsystem 24 will maintain control of the IPG as long an external power is 22 present.

If no external power 22 is available, the control program determines if a pulse is required or will be required within a predetermined short time. The need for a pulse can be determined by the state of a hardware circuit line or by an expired timer. If no pulse is needed, the main control program will increment any operating timers as required and perform any needed housekeeping functions before stopping the fast clock and returning to sleep mode. One skilled in the art will appreciate that the fast clock may have more than one rate. For example, a higher rate may be used for external telemetry.

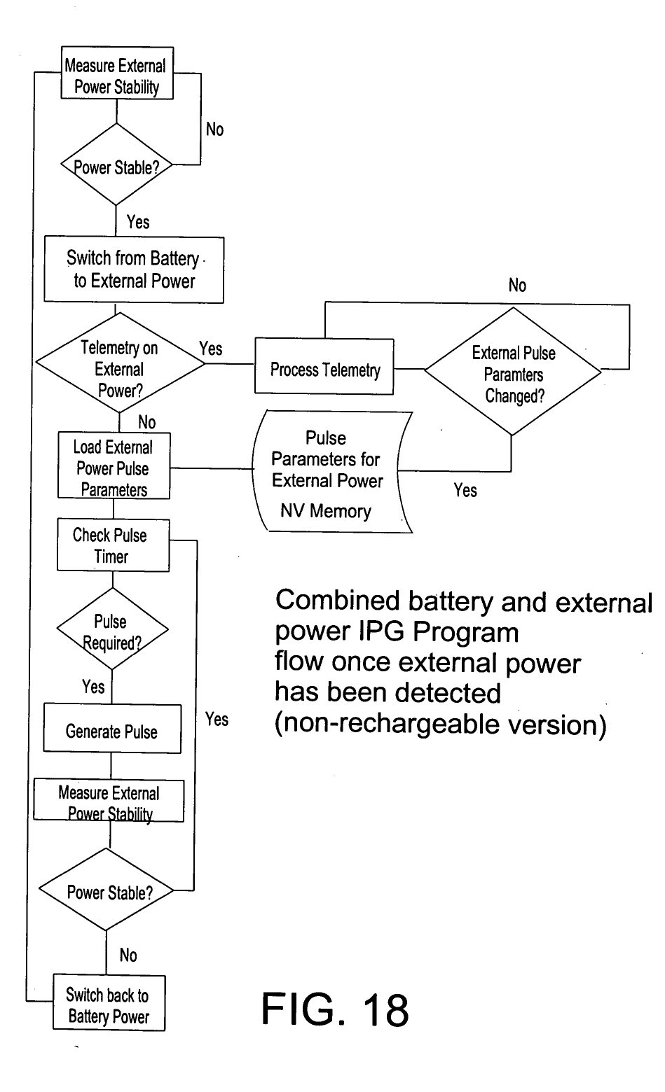

Shown in conjunction with FIG. 18 is a flowchart (for non-rechargeable version) which shows the process that runs after the system has detected the presence of external power (see Software Flowchart FIG. 17). This process first measures the stability of the external power and loops until the power is determined to be stable, only then will the system be switched from battery power to external power. If telemetry is detected, it is processed to extract the commands and change any pulse parameters in memory if required. If no telemetry is present, the process will load from non-volatile memory the pulse parameters for use when external power is present. Because conserving battery energy is not a concern on external power, the pulse output can be set to higher amplitude, duration and repetition rates. The process then assumes control of outputting pulses while periodically monitoring the stability of the external power. If the external power becomes unstable or is no longer present, the process will switch the system back to battery power.

For the IPG to deliver electrical pulses, the pulse generating unit charges up a capacitor and the capacitor is discharged when he control (timing) circuitry requires the delivery of a pulse. The unit uses pump-up capacitors to deliver pulses of larger magnitude than the potential of the batteries. The pump up capacitors are charged in parallel and discharged into the output capacitor series.

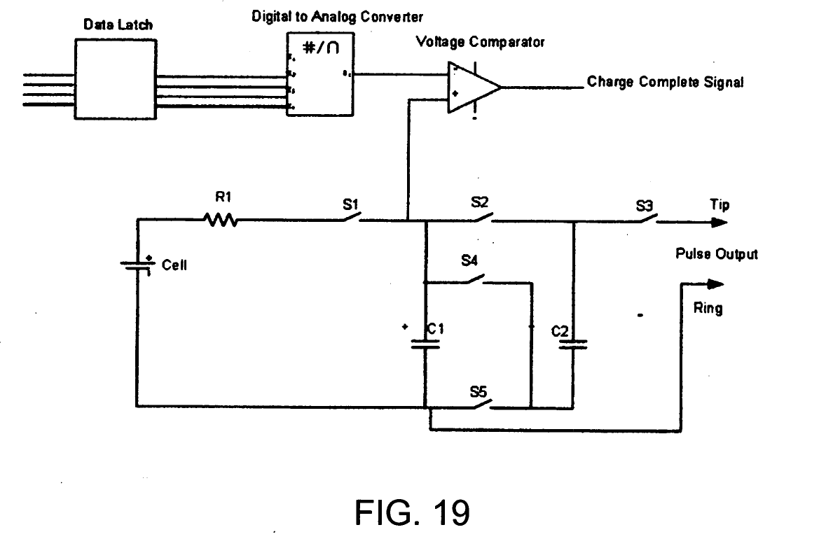

FIG. 19 shows a pulse generator output circuit using a charge pump implementation that produces variable output pulses with an amplitude of up to two time the system voltage. An arrangement of switches (implemented by transistors) is provided that allows the tank capacitors to be connected either in parallel for charging or in series for pulse output. The general equation for the pulse output voltage is:

$${V}_{Pulse} = 2 \cdot {V}_{System} \cdot \frac{{DAC}_{input}}{{2}^{DACbits}}$$

where:

DACbits is the number of input bits of the digital to analog converter, DACinput is the decimal reorientation of the digital word present at the input of the digital to analog converter (DAC)

For example, a system powered by a single Li-I cell with a nominal voltage of three volts may use a system voltage of 2.5 volts and thus have a maximum pulse amplitude of five volts. This is achieved by charging two “tank” capacitors (typically tantalum electrolytic type, 60 uF each or greater) in parallel to a voltage value of one half the required pulse amplitude. The tank capacitors are then placed in series and discharged through the lead electrodes for a set time period.

During charging, the tank capacitor voltage is compared to a reference voltage using a voltage comparator that provides a logic high signal when the tank voltage is equal to or greater than the reference voltage. This signal is used by the system to stop the charging process and initiate the pulse output process. In this case, the reference voltage is generated by a four-bit digital to analog converter. This provides 16 set points from zero to the system voltage of 2.5 volts. Thus the final pulse output may be set from zero to five volts in increments of 0.333 Volts. Using a DAC with more input bits will provide a corresponding increase in available resolution.

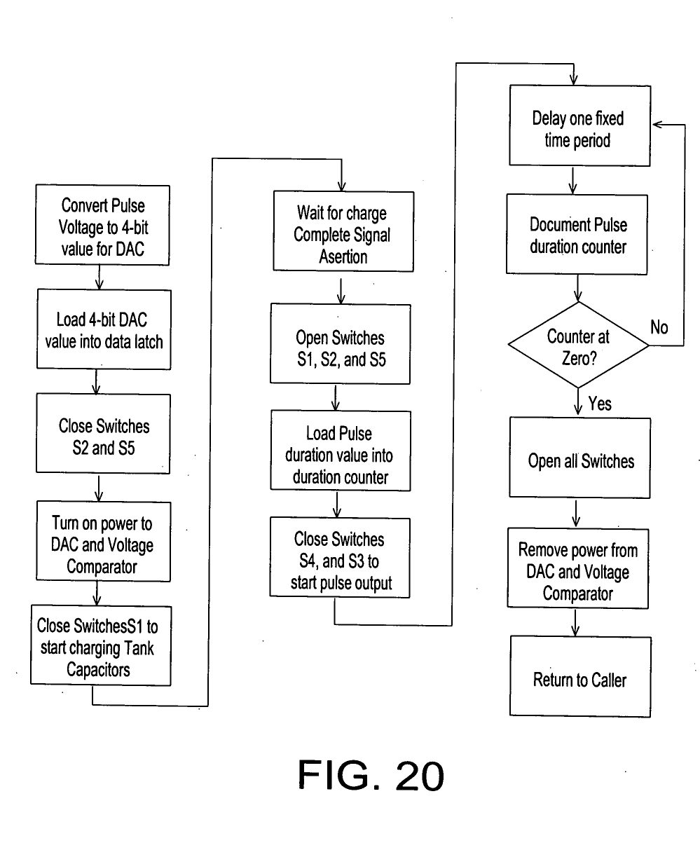

FIG. 20 shows the system software flow when a pulse is called for. In the example, with a 4-bit DAC, the software will multiply the requested pulse voltage by three and round to the nearest integer to get the decimal value of the DAC setting. The binary equivalent will then be set in the data latch. The system will set the switches to place the tank capacitors in parallel for charging and then apply power to the DAC and voltage comparator. The system then closes the switch to start the charging process and waits for the signal that the charging is complete. The switches are then configured for pulse output and the pulse duration counter is loaded with a value determined by the programmed pulse width. After a delay period, the counter is decremented and its value checked for zero. If it's not zero, the counter is decremented again after another delay. This repeats until the counter reaches zero when all the switches are opened to stop the output pulse and power is removed from the DAC and comparator. The software routine then returns control to its caller.

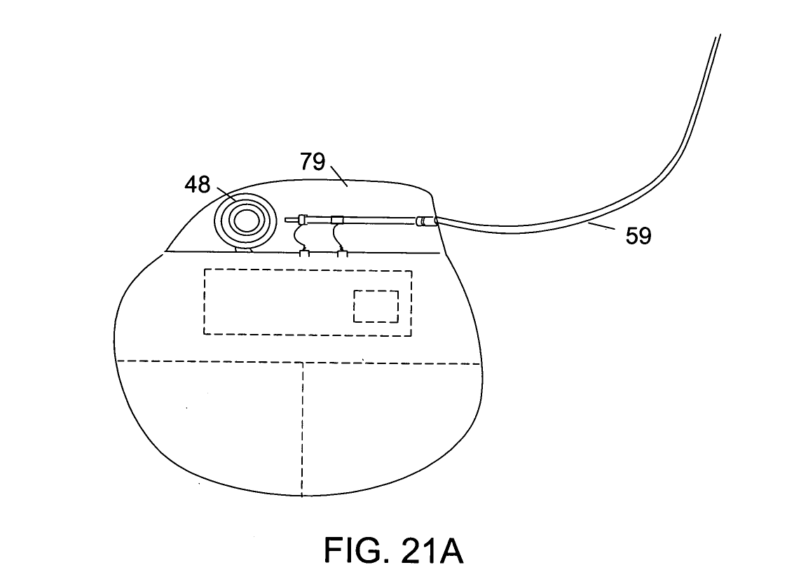

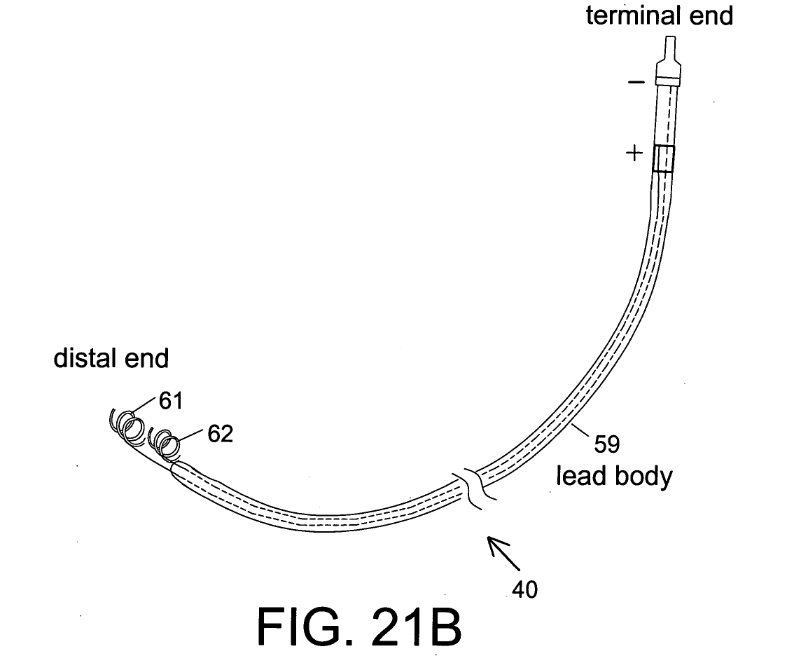

It will be clear to one skilled in the art, that different other embodiments of the above disclosure can be practiced. For example, as shown in conjunction with FIG. 21, the coil can be in the header portion, instead of being around the titanium case. It will also be clear to one skilled in the art that even though one channel of stimulation is shown in the drawings, for some application of the invention, the stimulation will be multi-channel. In some cases stimulation pulses may be provided via one lead, and blocking pulses may be provided via a second lead.

Shown in conjunction with FIG. 21B is a representative lead that is used with the system of the current invention. The electrodes 61,62 at the distal end of the lead will be adapted to contact different nerve or muscle tissue depending on the specific application.

### APPLICATIONS

As previously mentioned, the stimulation system of the current invention is particularly useful for neuro and muscle stimulation applications where the stimulation energy demands are significant. Without limitation, some of these indications and applications where the system of the current invention is particularly useful are mentioned below:

### Dorsal Column Stimulation for Pain and Refractory Angina

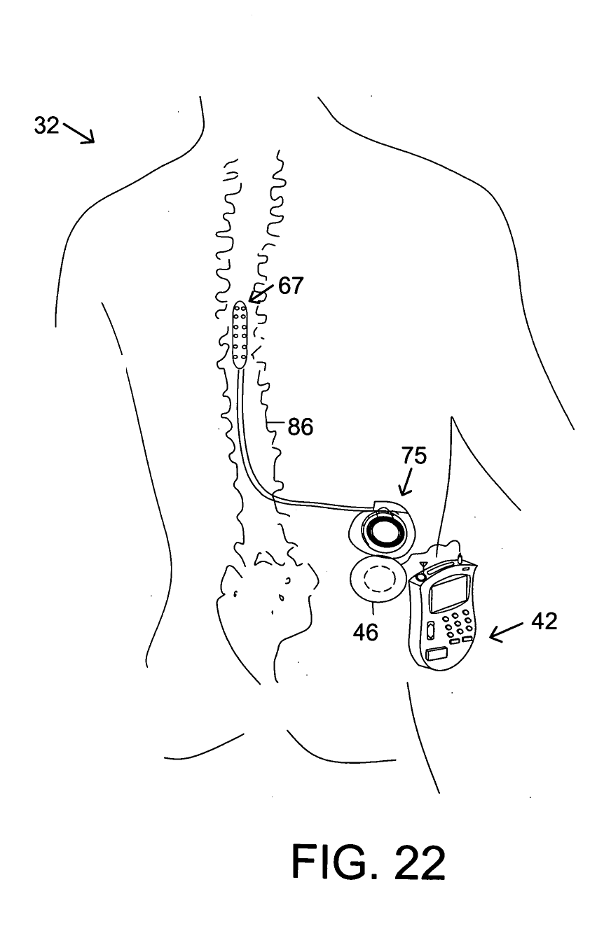

Shown in conjunction with FIG. 22, is a system of the current invention for spinal cord stimulation application. As shown in the figure, an electrode array 67 comprising a number of electrodes is implanted in the epidural space of the spinal cord. The distal end, which comprises the stimulating electrodes, may be paddle shaped as shown in FIG. 22, or alternatively may be cylindrical in shape. Stimulation pulses are provided via an implantable stimulator 75. The implantable stimulator 75 may provide pulses from an implanted power source, or the power and data may be provided from an external stimulator, via an external primary coil 46 which is inductively coupled to an implanted secondary coil 48.

Generally in patients, reliable and convenient stimulation is provided from the implanted power source. If the stimulation thresholds increase to a point where the drain of battery becomes significant, the external stimulator becomes a better alternative. Advantageously, in the system and method of this invention the patient and physician have the flexibility of using an implanted power source or an external power source.

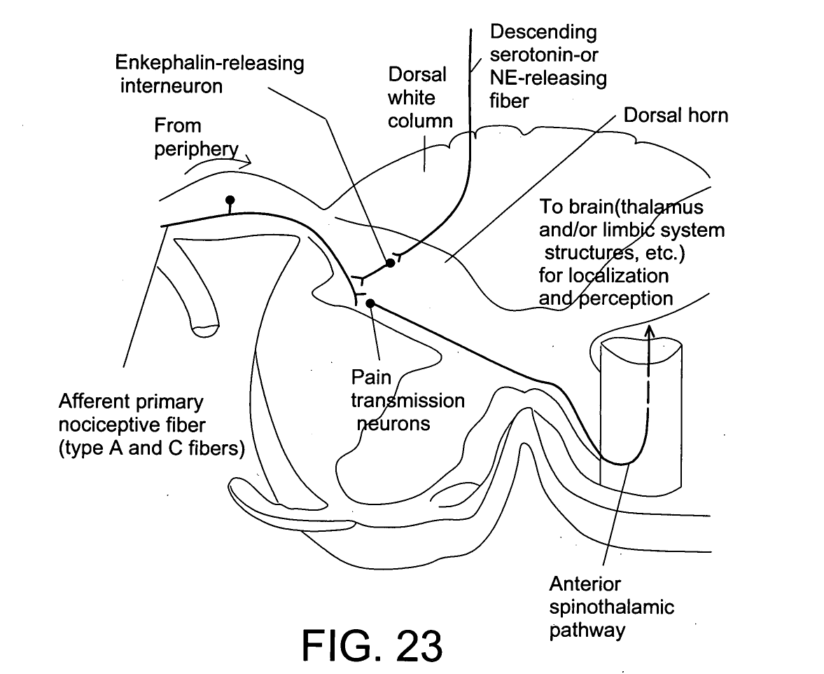

Spinal cord stimulation to provide pain relief is partially based on the gate theory of pain, which is explained in conjunction with FIG. 23.

In the body, natural neural mechanisms exist to modulate pain transmission and perception. The gate control theory of pain suggests that:

1) A pain “gate” exists in the dorsal horn (substantia gelatinosa) where impulses from small unmyelinated pain fibers and large touch (A beta) fibers enter the cord.

2) If impulses along the pain fibers outnumber those transmitted along the touch fibers, the gate opens and pain impulses are transmitted. If the reverse is true, the gate is closed by enkephalin-releasing interneurons in the spinal cord that inhibit transmission of both touch and pain impulses, thus reducing pain perception.

When type A delta and type C pain fibers transmit through to their transmission neurons in the spinothalmic pathway, pain impulses are transmitted to the cerebral cortex. Descending control of pain transmission (analgesia) is mediated by descending central fibers that synapse with small enkephalin-releasing interneurons in the dorsal horn that make inhibitory synapses with the afferent pain fibers. Activation of these interneurons inhibits pain transmission by preventing their release of substance P.

It has been found that (1) threshold stimulation of the large touch fibers results in a burst of firing in the substantia gelatinosa cells, followed by a brief period of inhibited pain transmission (it does close the pain “gate”), and (2) it has been amply proven that direct stimulation, or even transcutaneous electrical nerve stimulation (TENS), of dorsal column (large-diameter touch) fibers does provide extended pain relief.

It has been known that our natural opiates (beta endorphins and enkephalins) are released in the brain when we are in pain and act to reduce its perception. Hypnosis, natural childbirth techniques, morphine, and stimulus-induced analgesia all tap into these natural-opiate pathways, which originate in certain brain regions. These regions, which include the periventricular gray matter of the hypothalamus and the periaqueductal gray matter of the midbrain, oversee descending pain suppressor fibers that synapse in the dorsal horns. When transmitting, these fibers (most importantly some from the medullary raphe magnus) produce analgesia, presumably by synapsing with opiate (enkephalin) releasing interneurons that in turn actively inhibit forward transmission of pain inputs (FIG. 23). The mechanism of this inhibition appears to be that enkephalin blocks Ca²⁺ influx into the sensory terminals, thereby blocking their release of substance P. However, this is only one mechanism of pain modulation. A variety of other neurotransmitter receptor systems in the dorsal horn also regulate pain perception.

### Occipital Nerve Stimulation for Chronic Headaches, Transformed Migraine, and Occipital Neuralgia

Another nerve stimulation application where the system and method of the current invention is particularly useful is for occipital nerve stimulation to provide therapy or alleviate symptoms of chronic headaches, transformed migraine, and occipital neuralgia.

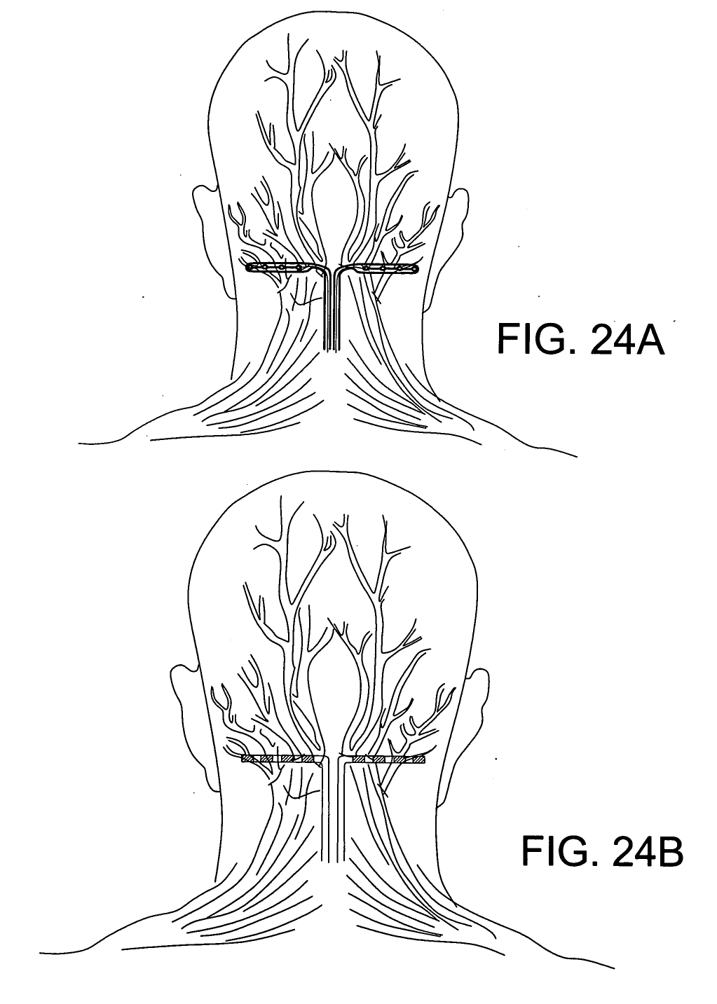

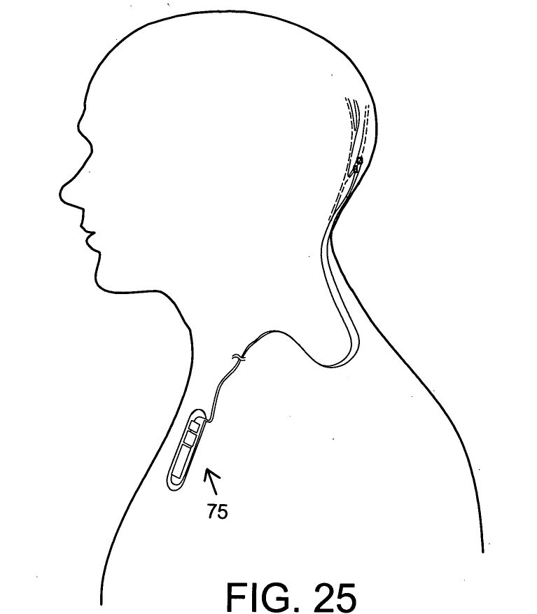

Shown in conjunction with FIGS. 24A and 24B is the placement of the electrode array for providing electrical pulses to the occipital nerves. A pair of paddle leads (FIG. 24A) or a pair of cylindrical leads (24B) are implanted subcutaneously in the back of the head. Alternatively, a single paddle lead or a single cylindrical lead may also be used for the electrode array. The terminal end of the lead(s) is tunneled to a convenient location for implantable stimulator pocket formation and implantation as is well known in the art (FIG. 23).

Medical and clinical studies have shown excellent clinical therapeutic efficacy by providing pulsed electrical stimulation to occipital nerves. Because of the energy output required to provide the therapy, the service life of a typical implantable pulse generator (with non rechargeable battery) is only 2-4 years. Because of this, the implantable stimulator system and method disclosed in this application would be particularly suitable for providing electrical pulses to occipital nerves to provide therapy for chronic headaches, transformed migraines, and occipital neuralgias. Applicant's co-pending U.S. patent application Ser. No. ______ provides more details regarding providing electrical pulses to occipital nerves to provide said therapy.

### Afferent Vagus Nerve Stimulation/Blocking to Provide Therapy for Central Nervous System (CNS) Disorders

Selective afferent stimulation/neuromodulation of vagus nerve(s) is known to provide therapy for epilepsy and severe depression. Additionally, chronic and intermittent afferent stimulation of vagus nerve(s) has also shown efficacy for bulimia/eating disorders, Alzheimer's disease, autism, chronic headaches/migraines, anxiety disorders and obsessive/compulsive disorder, and Parkinson's disease/essential tremor.

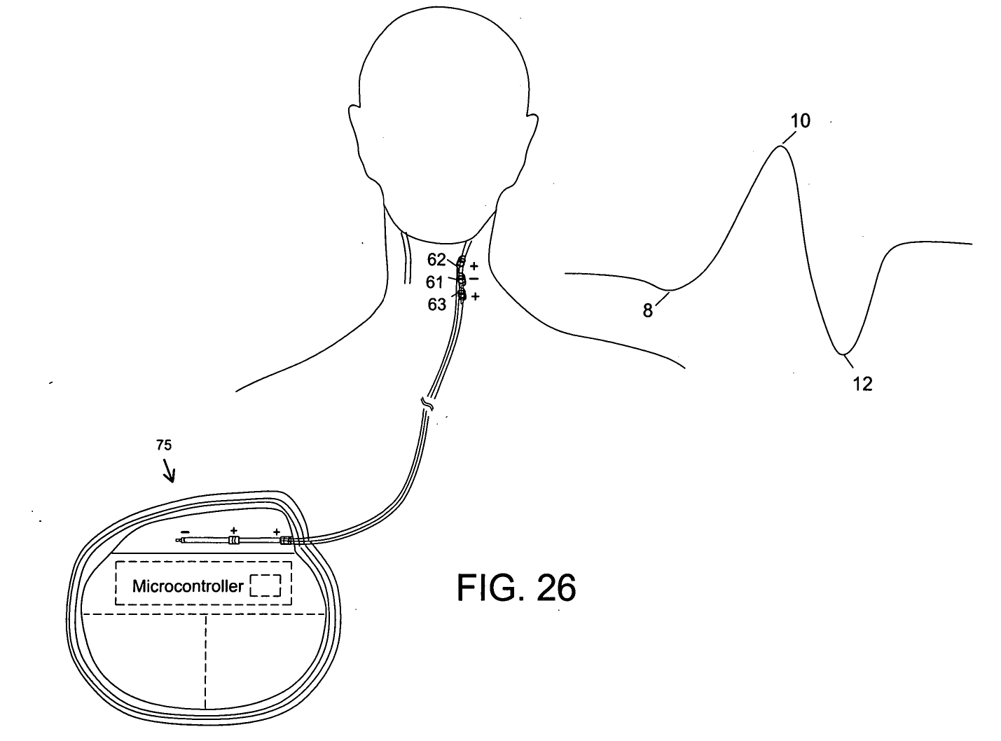

Shown in conjunction with FIG. 26, a lead which is in electrical connection with pulse generator, and has electrodes on the distal end which are adapted to wrap around the vagus nerve, typically the left vagus nerve. The vagus nerve(s), which runs in the carotid sheath, is typically isolated at the cervical level for placement of the electrodes around the vagus nerve(s). Alternatively, the electrodes can be attached around the level of diaphragm, either just above the diaphragm or just below the diaphragm.

To deliver therapy, electrical pulses are provided to vagus nerve(s) twenty-four hours per day, seven days a week. Stimulation is provided in a continuous intermittent manner, i.e. ON for a few minutes and OFF for a few minutes. The effects of therapy are cumulative over a period of time, usually months. Unlike cardiac pacing where a pulse is provided approximately once per second, the pulses to the vagus nerve(s) are provided at a repetition rate of approximately 20-50 pulses per second. Furthermore, in some embodiments additional blocking pulses may be provided to selected branches to minimize the side effects. For these reasons, vagus nerve(s) stimulation can be very demanding for an implanted non-rechargeable power source.

Advantageously, the systems and method disclosed in this application, is ideally suited for chronic intermittent vagus nerve(s) stimulation/blocking to provide therapy for neurological and neuropsychiatric disorders.

Applicant's other U.S. patents listed below also disclose details regarding afferent neuromodulation of vagus nerve(s) to provide therapy or alleviate symptoms of CNS disorders.

| USPN and date | Title |
|---|---|
| USPN 6,208,902 | Apparatus and method for adjunct (add-on) |
| 3/21/2001 | therapy for pain syndromes utilizing an |
| | implantable lead and an external stimulator. |
| USPN 6,356,688 | Apparatus and method for adjunct (add-on) |
| 3/12/2002 | therapy for depression, migraine, neuro- |
| | psychiatric disorders, partial complex |
| | epilepsy, generalized epilepsy and invol- |
| | untary movement disorders utilizing an |
| | external stimulator. |
| USPN 6,205,359 | Apparatus and method for adjunct (add-on) |
| 3/20/2001 | therapy of partial complex epilepsy, |
| | generalized epilepsy and involuntary |
| | movement disorders utilizing an external |
| | stimulator. |

### Deep Brain Stimulation

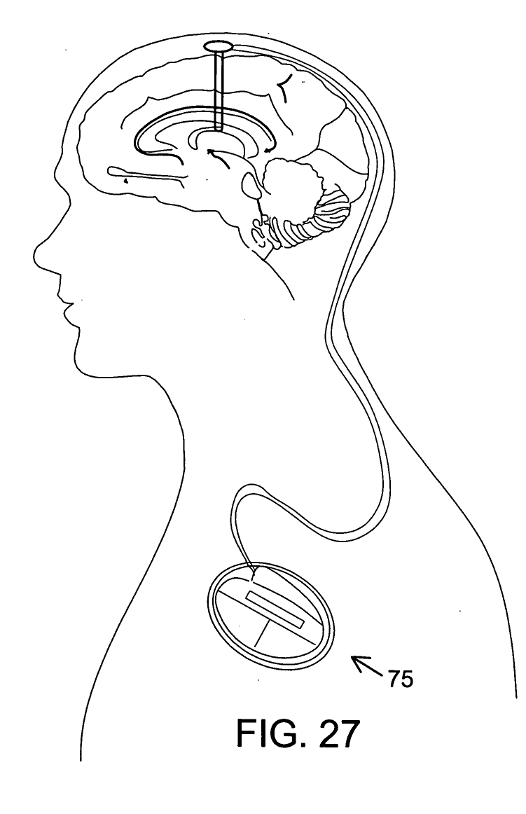

Another application for the systems and method of the current invention is for applying deep brain stimulation (DBS) to subthalamic nucleus or other deep brain structures. Subthalamic nucleus stimulation by means of permanently implanted brain electrodes (shown in FIG. 27), is a very effective therapy for all the cardinal features of Parkinson's disease. DBS has also been found to be successful in treating a variety of other brain-controlled disorders.

Generally, such treatment involves placing a DBS type lead through a burr hole drilled in the patient's skull. Following that, the lead is placed utilizing functional stereotactic brain surgery for applying appropriate stimulation through the lead. The placement portion of the treatment is very critical. The terminal portion of the lead is tunneled to a subcutaneous pocket where it is connected to the pulse generator, which is implanted in a pocket either subcutaneously or submuscularly.

### Vagal Nerve(s) Blocking to Provide Therapy for Obesity

Another application for the systems and method of this invention is to provide vagal nerve(s) blocking to provide therapy for obesity. The blocking of vagal nerve tissue may be one of DC anodal block, Wedenski block, or collision block.

Because of the high frequency of electrical pulses that may be involved for nerve blocking, this application is very demanding on the energy supply of the implanted pulse generator. Advantageously, the system and method of this invention is ideally suited for this type of application.

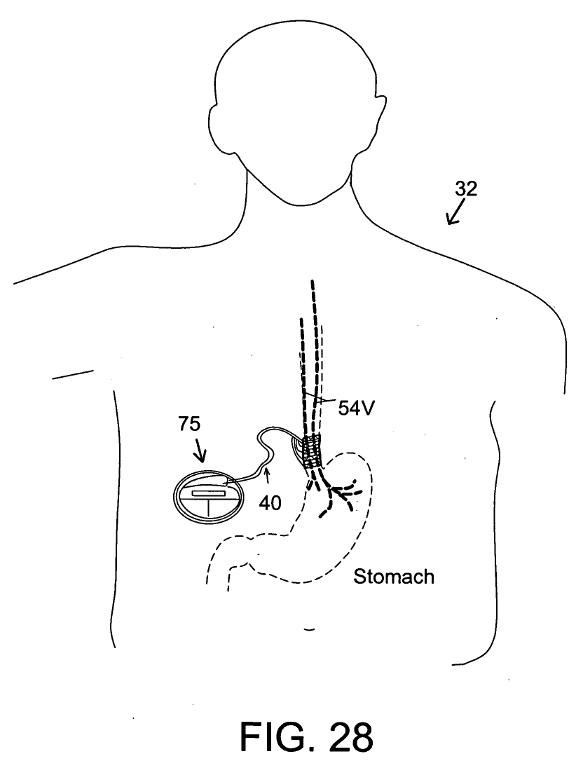

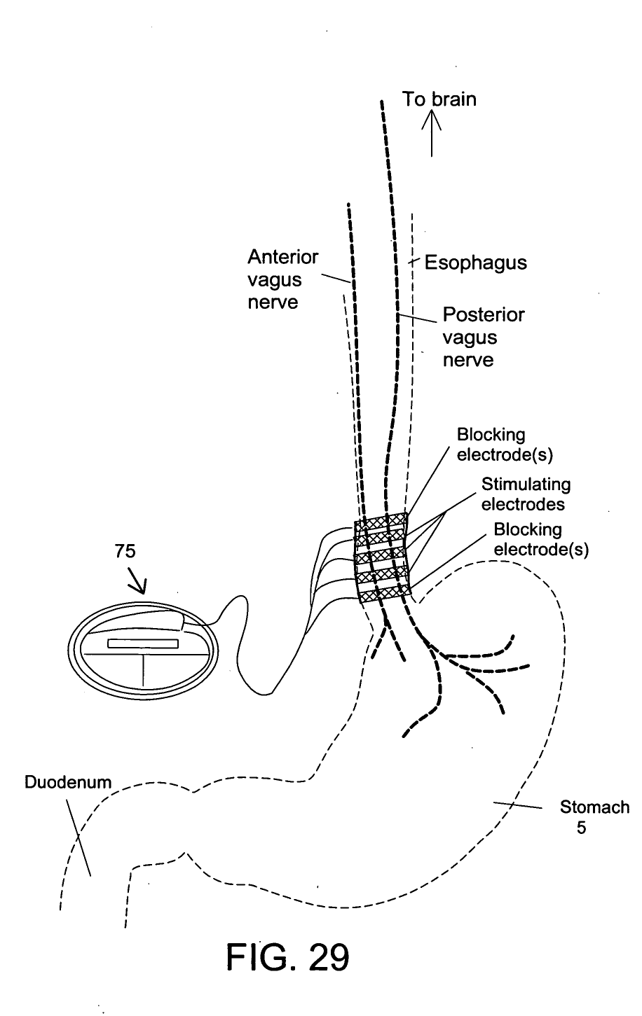

Shown in conjunction with FIGS. 28 and 29, multiple band electrodes may be wrapped around the esophagus, which ensures providing electrical pulses to the vagus nerves 54V, around the level of the diaphragm. As also depicted in FIG. 28, the multiple electrodes are connected to an implantable stimulator 75 via a lead 40. In this embodiment, the blocking pulses are provided to vagal nerves 54V via an external stimulator or via the power source of the implanted pulse generator.

Applicant's other co-pending patent applications listed below disclose more details on the methodology.

| No. and date | Title |
|---|---|
| 11/032652 | Method and system for vagal blocking and/or |
| Jan. 8, 2005 | vagal stimulation to provide therapy for |
| | obesity and other gastrointestinal disorders |
| | using implanted stimulus-receiver and an |
| | external stimulator. |
| 11/047232 | Method and system for vagal blocking with |
| Jan. 31, 2005 | or without vagal stimulation to provide |
| | therapy for obesity and other gastoin- |
| | testinal disorders using rechargeable |
| | implanted pulse generator. |

### Sympathetic Stimulation to Provide Therapy for Obesity

Another application for the system and method of this invention is to provide sympathetic stimulation to provide therapy for obesity. Sympathetic stimulation may be provided to celiac ganglion. More details are provided in Applicant's copending application Ser. No. 11/032,599 filed Jan. 08, 2005 entitled 41 Method and system to provide therapy for obesity and other medical disorders, by providing electrical pulses to sympathetic nerves or vagal nerve(s) with an external stimulator.

### Sacral Nerve(s) Modulation to Provide Therapy or Alleviate Symptoms of Urinary Incontinence

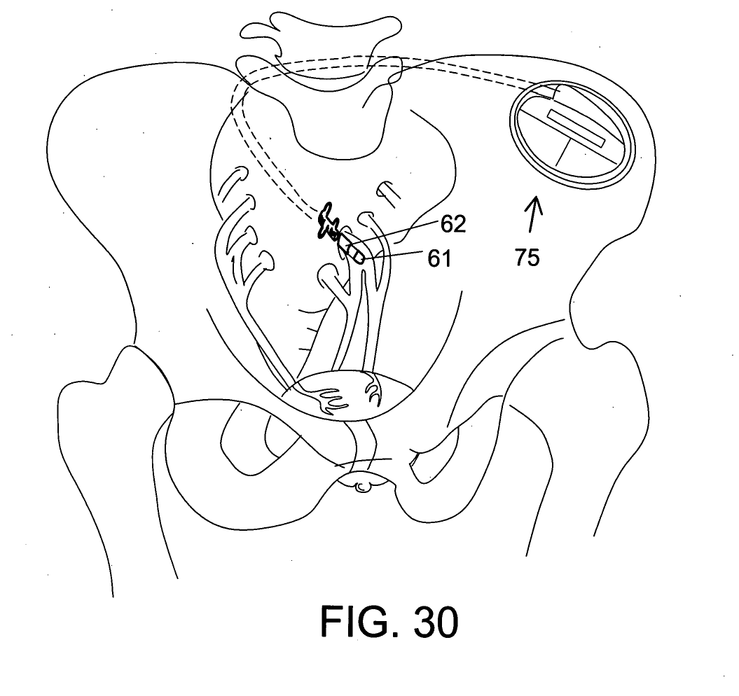

Another application for the systems and method of this invention is sacral nerve(s) modulation to provide therapy for urinary urge incontinence and other urological disorders. As shown in conjunction with FIG. 30, a lead comprising at least one electrode is implanted in one of the sacral foramen. The lead is tunneled subcutaneously, and the terminal end is connected to pulse generator means. Advantageously, with the systems and method of this invention, electrical pulses to the sacral plexus can be provided via an external stimulator or continuously via the implanted power source, according to a program stored in the memory.

Applicant's co-pending U.S. patent application Ser. No. 10/192,961 (filed on Jul. 16, 2002) entitled “Electrical stimulation adjunct (add-on) therapy for urinary incontinence and urolgoical disorders using implanted lead stimulus-receiver and an external pulse generator” provides more details regarding modulation of sacral plexus.

### Application to Erectile Dysfunction (ED)

Another application for the systems and method of this invention is to provide electrical stimulation of cavernous nerve(s) to provide treatment for erectile dysfunction,and electrical stimulation of nerves or nerve bundle in the sacral or pelvic region to provide therapy for chronic pelvic pain.

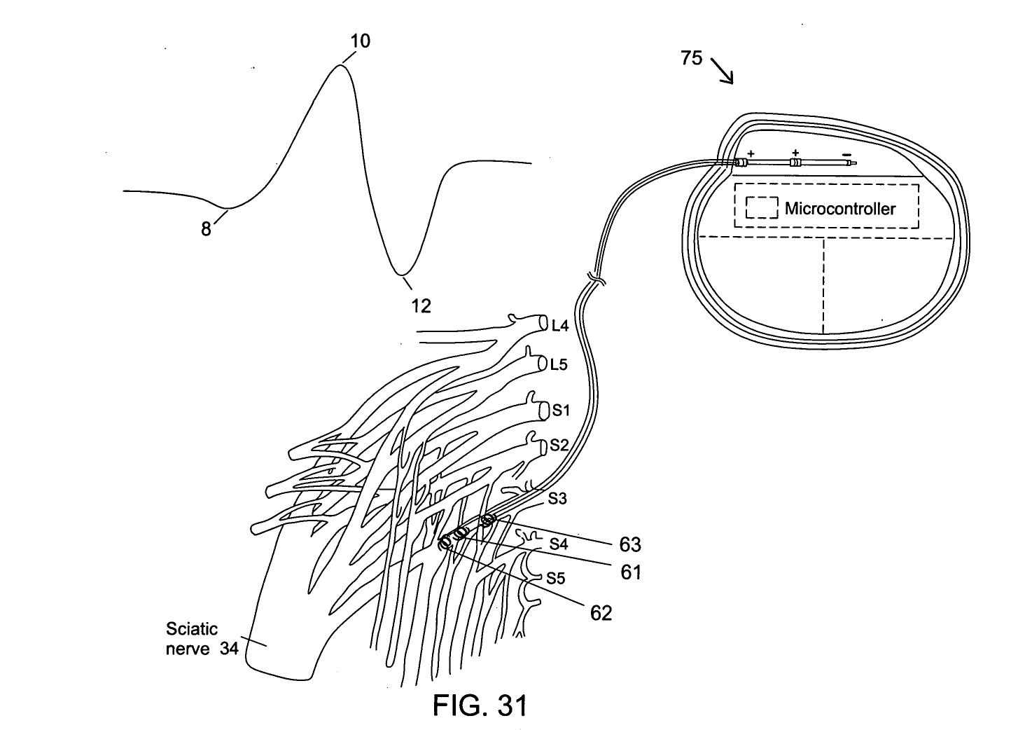

Shown in conjunction with FIG. 31, a lead is implanted with the electrodes in contact with appropriate nerve(s) in the sacral or pelvic region. The terminal end of the lead is tunneled subcutaneously and connected to a pulse generator 75, which is then implanted subcutaneously or submuscularly, as is known in the art.

Advantageously, electrical pulses can be provided either from an external stimulator or from an internal power source.

### Gastric Myo-Electrical Pacing to Provide Therapy for Obesity and Eating Disorders

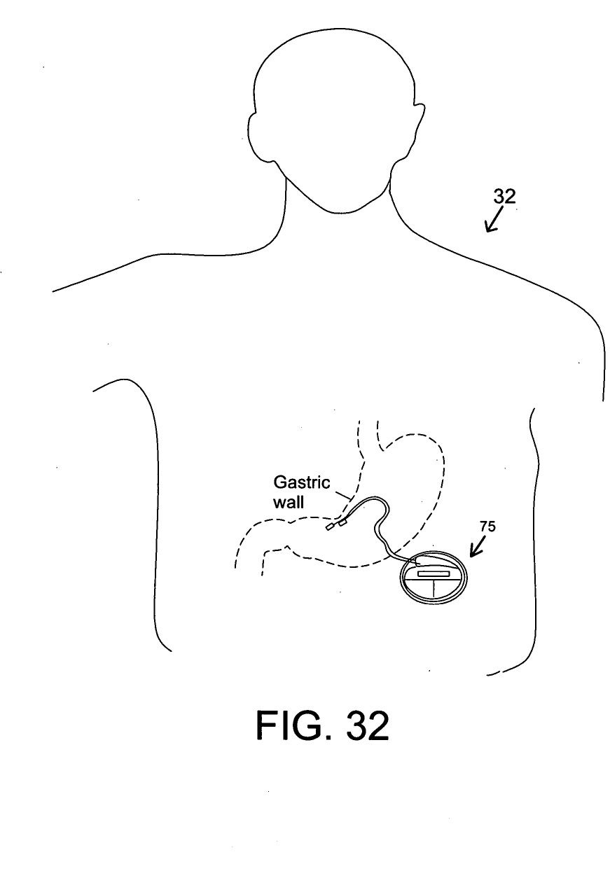

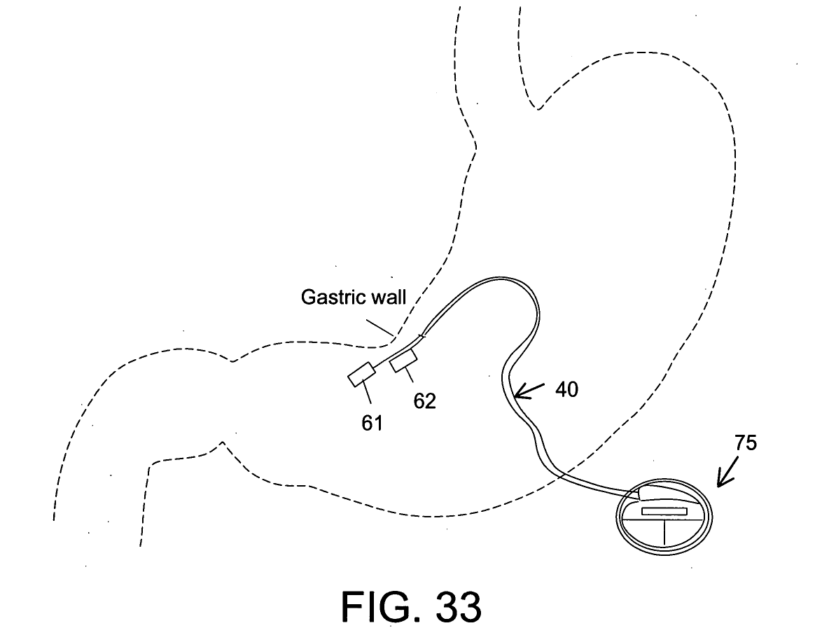

Yet, another application for the systems and method of this invention is to provide gastric myo-electric pacing therapy for obesity and eating disorders. Shown in conjunction with FIGS. 32 and 33, the electrodes on distal end of a lead 40 are implanted in gastric muscle tissue, typically utilizing laproscopic surgery. The terminal end of the lead 40 is tunneled subcutaneously in the usual manner and connected to an implanted stimulator 75, which is also implanted subcutaneously. By stimulating the stomach wall with the system described in this disclosure, using a site and frequency which competes with the intrinsic rhythm, the normal gastric motility is interfered with, and generally a decrease of normal gastric motility occurs. The stomach is emptied less efficiently. With the stomach not emptying as efficiently, satiety signals which are sent to the brain (via the vagus nerves), make the patients feel less hungry. With the capacity to handle less food through the gastrointestinal (GI) tract, and at the same time the patients feeling less hungry, therapy is provided for obesity and weight loss.

In contrast with nerve tissue stimulation, the pulse width needed to stimulate gastric muscle is significantly wider. This provides a significant drain on the power source. Advantageously, with the system and method of this invention the electrical pulses to the gastric wall may be provided via an external stimulator or via an implanted pulse generator.

The present invention may be embodied in other specific forms without departing from the spirit or essential attributes thereof. It is therefore desired that the present embodiment be considered in all aspects as illustrative and not restrictive, reference being made to the appended claims rather than to the foregoing description to indicate the scope of the invention.

We claim:

1. An implantable stimulator for providing electrical pulses to nerve or muscle tissue(s), comprises a programmable implantable pulse generator means and a stimulus-receiver means, wherein said stimulus-receiver means is adapted to function in conjunction with an external stimulator, whereby said electrical pulses to nerve or muscle tissues are provided either using said implanted pulse generator means or said external stimulator.

2. An implantable stimulator for providing electrical pulses to nerve or muscle tissue(s) for treating medical disorders, comprising:
an implantable pulse generation means comprising a microcontroller, memory, circuitry, and a battery power source;
an implantable stimulus-receiver means comprising circuitry and a secondary coil, and capable of receiving electrical pulses from an external stimulator and providing said pulses to said nerve or muscle tissue(s); and
circuitry means for switching between said implanted pulse generator means and said implanted stimulus-receiver means.

3. An implantable stimulator of claim 2, wherein said electric pulses generated by an external stimulator are provided to said nerve or muscle tissue(s) by bypassing said implanted battery source of said implantable stimulator.

4. The implanted stimulator of claim 2, wherein said secondary coil is inside a titanium case of said implanted stimulator.

5. The implanted stimulator of claim 2, wherein said secondary coil is outside a titanium case.

6. The implanted stimulator of claim 5, wherein said secondary coil is outside a titanium case in one of the following manner:
i) placed on the front side of said titanium case with a magnetic shield between the said secondary coil and said titanium case;
ii) around a titanium case and enclosed in epoxy, or
iii) placed in the header portion of said titanium case.

7. The implanted stimulator of claim 2, wherein said battery is a non-rechargeable battery.

8. The implanted stimulator of claim 2, wherein said battery is a rechargeable battery.

9. The implanted stimulator of claim 2, wherein implanted stimulator can be one of: single channel stimulator, dual-channel stimulator, or multi-channel stimulator.

10. The implanted stimulator of claim 2, wherein said electrical pulses comprise rectangular and/or complex pulses, wherein said complex pulses comprises pulses which are configured to be one of non-rectangular, multi-level, biphasic, or pulses with varying amplitude during the pulse.

11. The implanted stimulator of claim 2, wherein said electrical pulses comprise at least one variable component from a group comprising pulse amplitude, pulse width, pulse frequency, on-time and off-time wherein:
a) pulse amplitude ranges from approximately 0.1 volt to 25 volts;
b) pulse width ranges from approximately 20 micro-seconds to 5 milli-seconds;
c) pulse frequency ranges from approximately 0 to 750 Hertz;
d) on-time and off-time ranges from approximately 5 seconds to 24 hours.

12. The implanted stimulator of claim 2, wherein said implanted stimulator is used for providing said electrical pulses to a vagus nerve(s) of a patient for afferent vagus stimulation, to provide therapy or alleviate symptoms for at least one of epilepsy, depression, Parkinson's disease, bulemia, anxiety/obsessive compulsive disorders, Alzheimer's disease, autism, and neurogenic pain.

13. The implanted stimulator of claim 2, wherein said implanted stimulator is used for providing said electrical pulses to spinal cord of a patient, to provide therapy or alleviate symptoms for at least one of intractable pain or refractory angina.

14. The implanted stimulator of claim 2, wherein said implanted stimulator is used for supplying electrical pulses for deep brain stimulation of a patient, to provide therapy or alleviate symptoms of neurological or neurospsychiatric disorders.

15. The implanted stimulator of claim 2, wherein said implanted stimulator is used for providing said electrical pulses to gastric nerves or gastric wall of a patient to provide therapy for obesity:

16. The implanted stimulator of claim 2, wherein said implanted stimulator is used for providing said electrical pulses to provide therapy or alleviate symptoms for at least one of: rate control in atrial fibrillation; congestive heart failure; eating disorders; urinary urge incontinence; erectile dysfunction; occipital neuralgia or transformed neuralgia.

17. A system for providing electrical pulses to a body tissue(s) at one or more sites, comprising:
an implantable stimulator, wherein said implantable stimulator comprises a pulse generation means, a stimulus-receiver means, and a switching circuit means for operating selectively;
an external programmer means;
an external stimulator means adapted to work with said implanted stimulus receiver means;
at least one lead in electrical contact with said implantable stimulator; and
at least one electrode at a distal end of said at least one lead and adapted to be in electrical contact with said body tissue(s).

18. The system of claim 17, wherein said pulse generation means comprises a microcontroller, memory, pulse generation circuitry, and implanted battery, wherein said implanted battery may be rechargeable or non-rechargeable battery.

19. The system of claim 17, wherein said implantable stimulator comprises a coil which is inside a titanium case.

20. The system of claim 17, wherein said implantable stimulator comprises a coil which is outside a titanium case.

21. The system of claim 17, wherein said system is used for providing said electrical pulses to at least one of:
i) a vagus nerve(s) of a patient for afferent vagus stimulation, to provide therapy for one of Parkinson's disease, bulemia, anxiety/obsessive compulsive disorders, Alzheimer's disease, autism, and neurogenic pain;
ii) spinal cord of a patient to provide therapy or alleviate symptoms for intractable pain or refractory angina;
iii) deep brain of a patient to provide therapy or all alleviate symptoms for neurological or neurospsychiatric disorders;
iv) gastric nerves or gastric wall of a patient to provide therapy for obesity;
v) a vagus nerve(s) of a patient for efferent vagus stimulation for rate control in atrial fibrillation, or to provide therapy for congestive heart failure;
vi) sacral nerves or its branches in a patient, to provide therapy or alleviate symptoms of urinary urge incontinence;
vii) cavernous nerve(s) or branches of a patient to provide therapy for erectile dysfunction;
viii) occipital nerves or branches of a patient to provide therapy or alleviate symptoms for at least one of occipital neuralgia or transformed migraine.
